(19) United States
(12) Patent Application Publication
(10) Pub. No.: US 2023/0081686 A1
(43) Pub. Date: Mar. 16, 2023

(54) SYSTEM AND METHOD TO COMPENSATE FOR MOVEMENT DURING SURGERY

(71) Applicant: Epica International, Inc., San Clemente, CA (US)

(72) Inventor: Gregory William Stoutenburgh, San Clemente, CA (US)

(21) Appl. No.: 17/472,580

(22) Filed: Sep. 10, 2021

(51) Int. Cl. A61B 90/00; A61B 34/10; A61B 34/20

(52) U.S. Cl. CPC A61B 90/04; A61B 34/10; A61B 34/20; A61B 90/37; A61B 2034/105; A61B 2034/107; A61B 2034/2046; A61B 2034/2068; A61B 2090/374; A61B 2090/0418; A61B 2090/3762

(57) ABSTRACT

A system that compensates for movement during a surgical procedure on a patient includes a lidar array and a processor. The surgical procedure operates according to a surgical plan. The lidar array tracks the movement of the patient, a medical instrument, and/or a medical professional during the procedure. The processor modifies the surgical plan to compensate for one or more of the movements.

### BACKGROUND

Patients undergoing surgery often move during the procedure. Indeed, even the motion of the thorax during breathing is a type of movement during surgery. The surgeon and/or other medical professionals, e.g., nurses, technicians, etc., also make movements during surgery. In surgeries that involve precise techniques, such movements may interfere with planned, automated, and/or robotic surgical plans.

### BRIEF DESCRIPTION OF THE DRAWINGS

FIGS. 1A-1C are diagrams of systems that compensate for movement during a surgical procedure, according to embodiments of the present invention; and

FIG. 2 is a flowchart of a method that compensates for movement during a surgical procedure, according to an embodiment of the present invention.

Where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of embodiments of the invention. However, it will be understood by those of ordinary skill in the art that the embodiments of the present invention may be practiced without these specific details. In other instances, well-known methods, procedures, components, and circuits have not been described in detail so as not to obscure the present invention.

During surgery, tracking movement of the patient is important. This has been performed using human sight, cameras, computer vision, stereoscopic imaging using two cameras, and optical coherence tomography (OCT), just to name a few. See, e.g., Joel Williams, The Eyes Behind Surgical Robots, Photonics Spectra (April 2020); Marianne Andersen, Surgical Automation Gets More Precise Vision, Thanks to Multiple Data Sources, Robotics Business Review (Dec. 4, 2017). Human sight is often imprecise. Cameras, including stereoscopic cameras, may be blocked by the surgeons or medical professionals themselves and encounter shadows that reduce their efficiency. These cameras also often require the use of fiducial markers (or fiducials) to keep track of the patient, the surgical site, and/or a medical or surgical instrument.

The inventor has developed a system and method to determine and compensate for movement occurring during a surgical procedure that operates according to a surgical plan. The system involves using a lidar array to track the motion of the patient, the medical professionals providing care for the patient, and the medical instruments used by the medical professionals during the surgical procedure. By tracking these objects, the system can determine how much movement has occurred and can adjust a surgical plan to compensate for the movement. This system overcomes the problems of shadowing and using fiducials in prior art systems.

In addition, prior systems have a limited field of view and may only be able to visualize specific points in that field of view. In contrast, the present invention expands the field of view so as to provide an understanding of the entire surgical area. Moreover, the present invention is able to compensate for dynamic motion. For example, the present invention may use artificial intelligence (AI) to track organ displacement at different places while the patient is breathing during a procedure. The present invention may also use AI to track the deformity caused by insertion of a surgical device, e.g., a needle, during a procedure. Thus the present invention is able to improve precision during a procedure.

Lidar is a detection system that works on the principle of radar, but uses light from a laser instead of radio waves. Lidar determines ranges by targeting an object with a laser and measuring the time for the reflected light to return to the receiver. A lidar array in two dimensions allows mapping and movement tracking of all the structures in the transmission field.

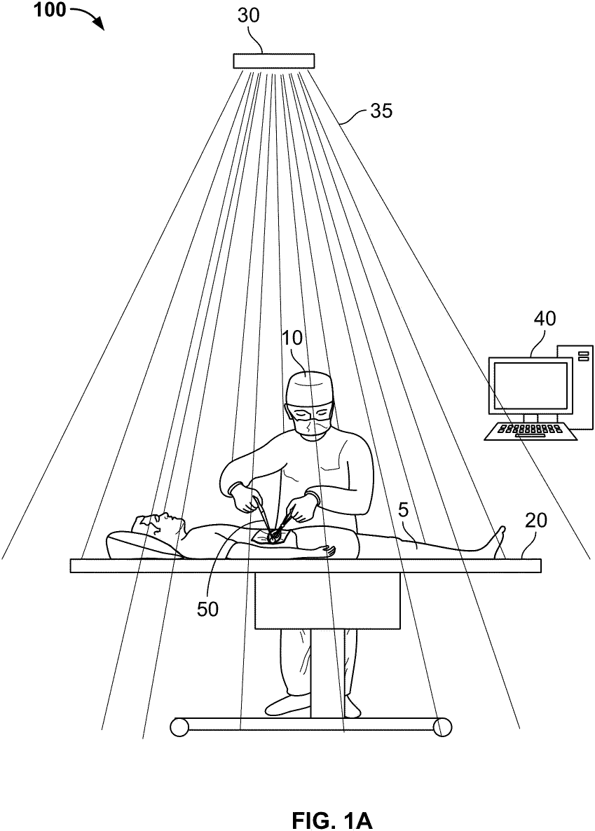

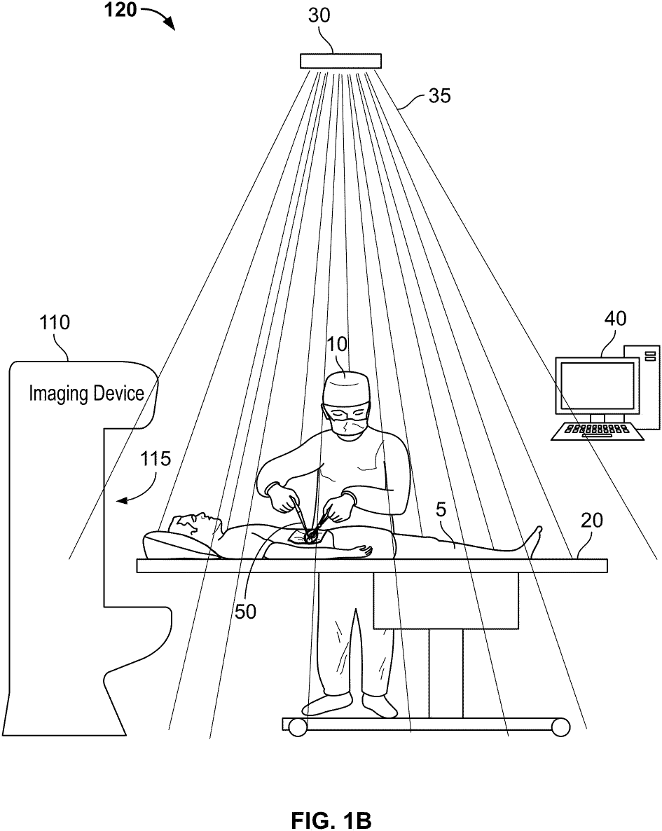

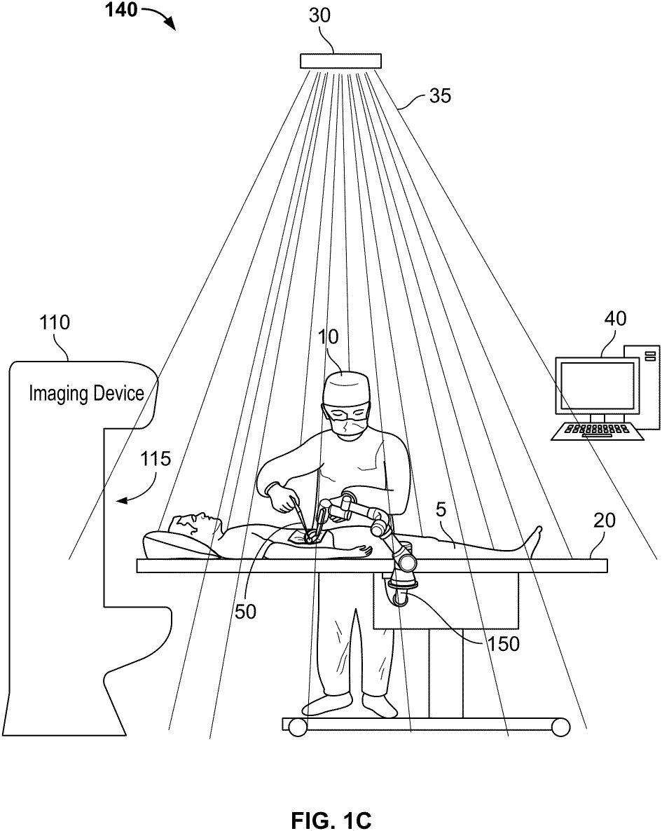

Reference is now made to FIGS. 1A-1C, which are diagrams of systems that compensate for movement during a surgical procedure, according to embodiments of the present invention. In FIG. 1A, system 100 includes bed 20, lidar array 30, and computer 40. In a typical setup, surgeon 10 uses medical or surgical instrument 50 to operate on patient 5, who is lying on bed 20. Surgical instrument 50 may include any medical or surgical instrument used during surgery, including a surgical guide that holds other surgical instruments. Examples of surgical instruments and surgical guides are disclosed in U.S. Pat. App. No. 16/160,575, which is incorporated herein by reference in its entirety, as if set forth fully herein. Examples of types of surgeries the system may be used in are cranial (including neuro, neural implants, brain, facial, and maxillary), ENT (ear-nose-throat), spinal procedures for cervical, thoracic, and lumbar regions; orthopedics (usually relating to hard, fixed structures); oncology (including interventional hybrid procedures); nephrology, urology, airway, obstetrics, gynecology, cardiovascular, and traumatology.

Lidar array 30 provides lasers in two dimensions and allows mapping and movement tracking of all the structures in transmission field 35. The array may typically use visible and infrared wavelengths. The current state of the art is 300 lasers per square degree, but the invention encompasses improvements in the state of the art. Thus, if the lidar array area is 20 degrees by 30 degrees, there may be 180,000 lasers or more. The lidar array looks at the whole surgical area, whereas imaging in the prior art looked at specific fiducials that were placed at the surgical site or on a surgical instrument. Lidar array 30 may image in 2D, but can also provide a 3D image with range information. A 3D image can be grayscale or in color if more than one wavelength of light is used. Lidar array 30 may control when light is emitted so it is able to directly measure range in each pixel based on time of flight to and from an object in a given pixel. Velocity may be measured either directly using the Doppler shift in frequency due to motion, or by multiple measurements of position. Coherent lidar is able to measure velocity very accurately. Lidar array 30 may be placed above bed 20, patient 5, and surgeon 10, but it may also be placed in other positions in the operating room if desired. Lidar array 30 communicates with computer 40.

Computer (or processor) 40 is representative of a computer system that may be used to develop and carry out a surgical plan. A surgical plan may include the steps of the surgical procedure, based on the types of implants and tools available to the surgeon. A preliminary plan may be made based on pre-operative diagnostic imaging such as CT or MRI taken days or weeks before the surgical procedure. This plan may be stored in computer 40. At the beginning of the procedure, a 3D scan of the patient on the surgical table may be taken using, e.g., CT or MRI. It is possible that things may have changed since the pre-operative imaging. This latest scan provides a foundation for where the patient is positioned, and the scan is able to detect the objects in the room based on, e.g., densities. Then a lidar scan (or lidar pattern) may be taken of the patient to register the shape of the body with the 3D shape from the CT or MRI. These scans are then registered in the computer 40.

During surgery, the inventive system knows the location of everything in the surgical area - the patient, the surgeon, the other medical professionals in the room, the surgical or medical instruments, etc. The system also keeps track of the number of surgical or medical instruments so that none are lost or left in the patient after surgery. Computer 40 may include surgical navigation software. Normal navigation software keeps the surgeon on the surgical plan but may not be able to modify the plan during surgery. Conventional navigation typically uses fiducial markers to track the surgery. In contrast, the present inventive system does not need to use fiducial markers, but tracks the movement of objects in the surgical area using the lidar array. In one embodiment, fiducial markers may still be used, and the system may also keep track of such markers. The surgical plan may also include the surgical path that the surgery is expected to follow.

Computer 40 may be, for example, a networked computer, a desktop computer, a laptop computer, a handheld computer, or a smartphone. It may operate as a standalone device or as part of a wired or wireless network. Such a network may be any type of communications network, including a public or private telephone (e.g., cellular, public switched, etc.) network and/or a computer network, such as a WAN (wide area network), MAN (metropolitan area network), or LAN (local area network) or the Internet or an intranet. The computer itself does not need to be present in the operating room, but needs to be able to communicate with lidar array 30.

Although lidar array 30 and computer 40 are shown as separate entities, lidar array 30 may incorporate a computer that can develop a surgical plan as well as modify the surgical plan to compensate for movements during surgery. In this way, it may be said that lidar array 30 tracks such movements and modifies or adjusts the surgical plan. Thus, even if lidar array 30 and computer 40 are separate entities, it may still be said that lidar array 30 tracks such movements and modifies or adjusts the surgical plan, even if such modification is actually performed by computer 40.

Moreover, lidar array 30 and computer 40 may include the ability to use AI techniques to recognize the patient and objects in the operating room, including surgeon 10 and surgical instrument 50, as well as to track the movements of such patient and objects. Such AI techniques may include various machine learning algorithms that learn over time to better distinguish the patient and objects, the types of objects, and the typical and atypical movements such patients and objects make. For example, the system may be taught how to identify certain structures or items. Over time, the system can learn the identity of other structures, as well as how the structures on which it was trained interact. Without AI, the knowledge of these structures would be static. In addition, safety checks can be built-in to the surgical procedure. If the system understands the steps of the procedure and plans them in advance, then the system may send out warnings if the surgeon skips a step or otherwise diverges too much from the plan. This aspect is good for teaching and training surgeons. And just as the system checks the surgeon’s steps, the system may also be able to validate movements and placements by a surgical robot or robotic arm, which is described below with respect to FIG. 1C.

During surgery, the patient may move - motion is normal and a necessary part of the procedure. In the present invention, such motion is dynamically and in real-time accounted for, and the system validates that the surgery is happening as planned. The system also allows the surgeon to watch him- or herself doing something that he or she cannot see. For example, the lidar knows where the tip of a surgical instrument, such as a drill bit in a drill, is. If computer 40 knows where the drill bit is supposed to hit, but the patient is breathing and thus the surgical area may be moving slightly, the computer can tell the surgeon to make adjustments based on such breathing or motion.

AI can be used to keep track of such dynamic motion. For example, as part of a training protocol, the system can observe a surgical area from different patients at full respiratory inflation and several values of partial respiratory inflation and can model the movement at such inflation levels, If there is a tumor in the abdomen or kidney, the system can predict how those body structures are moving without rescanning. In addition, the system may be used to predict the motion in the body – for example, how will the abdomen, muscles, etc. move. And then during surgery, the system may assess the ways the body in front of it is moving and breathing.

In FIG. 1B, system 120 includes the items in FIG. 1A plus imaging device 110. Imaging device 110 may perform X-rays, CT scans, MRI, and fluoroscopy. Imaging may be performed before or during the surgical procedure, or both. Imaging device 110 includes an analysis zone 115 suitable to house at least a portion of bed 20. Bed 20 typically has wheels, so that it may move into and out of analysis zone 115. One example of an imaging device is disclosed in U.S. Pat. No. 10,016,171, which is incorporated herein by reference in its entirety, as if set forth fully herein.

In FIG. 1C, system 140 includes the items in FIG. 1B plus surgical robot or robotic arm 150. Robotic arm 150 may assist surgeon 10 during surgery. Robotic arm 150 may hold surgical instrument 50 or other surgical or medical instruments, or may hold a surgical guide that in turn holds a surgical instrument, as described above. Robotic arm 150 may be controlled by computer 40. Examples of robotic arms are disclosed in U.S. Pat. App. No. 16/402,002, which is incorporated herein by reference in its entirety, as if set forth fully herein.

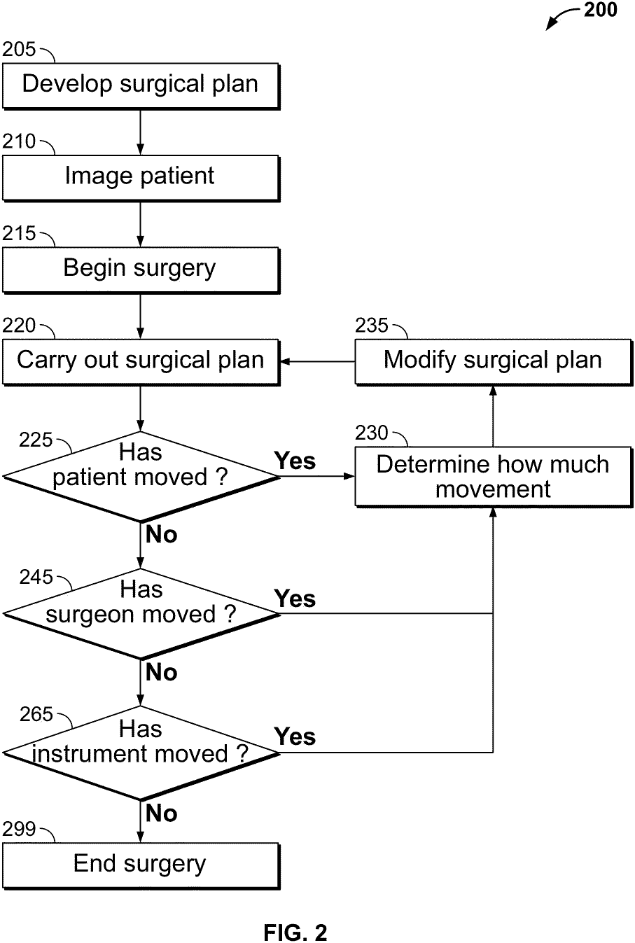

Reference is now made to FIG. 2, which is a flowchart showing a process 200 that compensates for movement during a surgical procedure, according to an embodiment of the present invention. In operation 205, a surgical plan is developed, as described above. In operation 210, the patient is imaged with an imaging device. While this imaging is shown as preoperative, it may also occur during the surgical procedure. The imaging may include X-rays, CT scans, MRI, and fluoroscopy. In operation 215, surgery begins. Such surgery may be manual, robotic/automatic, or a combination. In operation 220, surgeon 10 and/or surgical robot 150 carries out the surgical plan. In one embodiment, the navigation software running on computer 40 supplies the intervention point, angle, and direction (axis) of intervention to the robotic arm, which moves to place the surgical instrument at the correct point, angle, and direction.

During this time, lidar array 30 is monitoring the operating room, via transmission field 35. It is recognizing objects as patient, medical professionals, and surgical or medical instruments. Operation 225 asks whether the patient has moved. If so, operation 230 determines how much the patient has moved, and operation 235 modifies the surgical plan based on the patient’s movement. This modification may include changing the trajectory, angle, or direction of the instrument or the force with which the instrument is wielded. Other modifications may include adjusting the orientation of the patient, modifying the surgical path to avoid structures, and adjusting or changing the actual instrument to one that is thinner, sharper, etc. Similarly, operation 245 asks whether the surgeon or other medical professional has moved. If so, operation 230 determines how much the surgeon or other medical professional has moved, and operation 235 modifies the surgical plan based on that movement, making some or all of the modifications described above regarding the patient movement. Finally, operation 265 asks whether the surgical instrument has moved. If so, operation 230 determines how much the instrument has moved, and operation 235 modifies the surgical plan based on that movement, making some or all of the modifications described above regarding the patient movement. This loop of determining whether the patient, medical professional, or instrument has moved continues until there is no more movement, at which point the process continues to operation 299 to end the surgery.

Besides the operations shown in FIG. 2, other operations or series of operations are contemplated to determine how much movement occurs during a surgical procedure and compensate for it. For example, even though there may be movement, the movement may not require a modification of the surgical plan. In addition, as will be discussed below, there may be certain circumstances when fiducials are still used. In those circumstances, the lidar array can track the movement of the fiducial(s), and modify the surgical plan accordingly.

Moreover, the actual order of the operations in the flowchart in FIG. 2 is not intended to be limiting, and the operations may be performed in any practical order. For example, tracking of movement of medical professional and instrument may occur before that of the patient. More likely, however, the process monitors all three types of movements simultaneously or substantially simultaneously.

In sum, the invention uses a lidar array to track movement of objects and people in the operating room so as to modify or adjust the surgical plan based on the movements. It overcomes the problem of shadows that occur using a camera-based system. And it generally allows the surgery to be performed without using fiducial markers, although the invention may also track such fiducials if they are used. For example, in some cases, lidar may have difficulty penetrating sterile drapes if they are used during surgery. In such cases, fiducials may be used, and the lidar can track the movement of the fiducials. Because the invention tracks movement of objects in the operating room, it may also be used to count the surgical or medical instruments (and objects) so as to keep track of them at the end of surgery to prevent them from accidentally being left inside the patient.

Aspects of the present invention may be embodied in the form of a system, a computer program product, or a method. Similarly, aspects of the present invention may be embodied as hardware, software, or a combination of both. Aspects of the present invention may be embodied as a computer program product saved on one or more computer-readable media in the form of computer-readable program code embodied thereon.

The computer-readable medium may be a computer-readable storage medium. A computer-readable storage medium may be, for example, an electronic, optical, magnetic, electromagnetic, infrared, or semiconductor system, apparatus, or device, or any combination thereof.

Computer program code in embodiments of the present invention may be written in any suitable programming language. The program code may execute on a single computer or on a plurality of computers. The computer may include a processing unit in communication with a computer-usable medium, where the computer-usable medium contains a set of instructions, and where the processing unit is designed to carry out the set of instructions.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A system to compensate for movement during a surgical procedure on a patient in a surgical area, the procedure operating according to a surgical plan, the system comprising:
a lidar array configured to track the movement of one or more of the patient, a medical instrument, or a medical professional during the procedure; and
a processor configured to modify the surgical plan to compensate for one or more of the movements.

2. The system of claim 1, wherein the processor modifies a surgical path based on one or more of the movements.

3. The system of claim 1, wherein the processor further keeps track of the number of medical instruments in the surgical area.

4. The system of claim 1, wherein the lidar array tracks the movement of a fiducial marker in the surgical area.

5. The system of claim 1, further comprising an imaging device.

6. The system of claim 5, wherein the imaging device images the patient before the procedure.

7. The system of claim 6, wherein the imaging of the patient is a 3D scan.

8. The system of claim 5, wherein the imaging device images the patient during the procedure.

9. The system of claim 8, wherein the imaging device generates an image that is registered to the patient’s body.

10. The system of claim 5, wherein the imaging device comprises one or more of a computed tomography (CT) device, a magnetic resonance imaging (MRI) device, and a fluoroscopic imaging device.

11. A method to compensate for movement during a surgical procedure on a patient in a surgical area, the procedure operating according to a surgical plan, the method comprising:
tracking movement, using a lidar array, of one or more of the patient, a medical instrument, or a medical professional during the procedure; and
modifying the surgical plan to compensate for one or more of the movements.

12. The method of claim 11, further comprising modifying a surgical path based on one or more of the movements.

13. The method of claim 11, further comprising keeping track of the number of medical instruments in the surgical area.

14. The method of claim 11, further comprising tracking the movement of a fiducial marker in the surgical area.

15. The method of claim 11, further comprising imaging the patient before the procedure using an imaging device.

16. The method of claim 15, wherein the imaging of the patient is a 3D scan.

17. The method of claim 15, wherein the imaging device comprises one or more of a computed tomography (CT) device, a magnetic resonance imaging (MRI) device, and a fluoroscopic imaging device.

18. The method of claim 11, further comprising imaging the patient during the procedure using an imaging device.

19. The method of claim 18, further comprising registering an image from the imaging device to the patient’s body.

20. The method of claim 18, wherein the imaging device comprises one or more of a computed tomography (CT) device, a magnetic resonance imaging (MRI) device, and a fluoroscopic imaging device.
